(19) United States
(12) Patent Application Publication
(10) Pub. No.: US 2020/0248129 A1
(43) Pub. Date: Aug. 6, 2020

(54) SPERM SORTER AND SPERM SORTING METHOD

(71) Applicants: National Tsing Hua University, Hsinchu City (TW); Taipei Medical University, Taipei City (TW)

(72) Inventors: Fan-Gang Tseng, Hsinchu City (TW); Li-Chern Pan, Taipei City (TW); Jen-Kuei Wu, Hsinchu City (TW); Yung-Chin Tzeng, Hsinchu City (TW); Suei-Shen Wang, Hsinchu City (TW)

(73) Assignees: National Tsing Hua University, Hsinchu City (TW); Taipei Medical University, Taipei City (TW)

(21) Appl. No.: 16/439,685

(22) Filed: Jun. 12, 2019

(30) Foreign Application Priority Data
Feb. 1, 2019 (TW) 108104120

(51) Int. Cl. C12M 1/00; C12N 5/076; C12M 3/00; C12M 1/26

(52) U.S. Cl. CPC C12M 47/04; C12M 33/14; C12M 21/06; C12N 5/061

(57) ABSTRACT

A sperm sorter and a sperm sorting method are provided. The sperm sorter includes an inlet chamber, a swim up sorting chamber, a divergent channel, a recycling chamber and an outlet chamber. The swim up sorting chamber is communicated with the inlet chamber. The swim up sorting chamber is communicated between the inlet chamber and the divergent channel. The divergent channel has an entrance terminal close to the swim up sorting chamber and an exit terminal away from the swim up sorting chamber. A width and a depth of the entrance terminal are respectively less than a width and a depth of the exit terminal. The recycling chamber is communicated with the exit terminal of the divergent channel. The outlet chamber is communicated with a portion of the divergent channel between the entrance terminal and the exit terminal.

### CROSS-REFERENCE TO RELATED APPLICATION

This application claims the priority benefit of Taiwan application serial no. 108104120, filed on Feb. 1, 2019. The entirety of the above-mentioned patent application is hereby incorporated by reference herein and made a part of this specification.

### BACKGROUND

### Technical Field

The present disclosure relates to a sperm sorter and a sperm sorting method.

### Description of Related Art

Infertility has become one of the common problems having impacts on families in modern society. Various artificial fertilization methods have been developed for addressing such problem. For instance, current artificial fertilization methods include intrauterine insemination (IUI), in vitro fertilization (IVF) and intracytoplasmic sperm injection (ICSI) and so forth. Motility and amount requirements of sperms used for various artificial fertilization methods are different. Thereby, a method for precisely sorting sperms by properties of sperms (e.g., motility) is important in the art.

### SUMMARY

Accordingly, the present disclosure provides a sperm sorter and a sperm sorting method that are capable of sorting sperms for different artificial fertilization methods.

According to some embodiments, a sperm sorter includes an inlet chamber, a swim up sorting chamber, a divergent channel, a recycling chamber and an outlet chamber. The swim up sorting chamber is communicated with the inlet chamber. The swim up sorting chamber is communicated between the inlet chamber and the divergent channel. The divergent channel has an entrance terminal close to the swim up sorting chamber and an exit terminal away from the swim up sorting chamber. A width and a depth of the entrance terminal are respectively less than a width and a depth of the exit terminal. The recycling chamber is communicated with the exit terminal of the divergent channel. The outlet chamber is communicated with a portion of the divergent channel between the entrance terminal and the exit terminal.

In some embodiments, the sperm sorter further includes a filtering structure. The filtering structure is disposed in the inlet chamber. A semen sample entering the inlet chamber is filtered by the filtering structure before flowing into the swim up sorting chamber.

In some embodiments, the sperm sorter further includes an outlet channel and a block. The outlet channel is communicated between the exit terminal of the divergent channel and the recycling chamber. The block is disposed in the outlet channel.

In some embodiments, the block protrudes from a top surface of the outlet channel into the outlet channel. A thickness of one end of the block close to the divergent channel is less than a thickness of another end of the block away from the divergent channel.

In some embodiments, the divergent channel includes a plurality of micro-channels. The plurality of micro-channels are arranged to be substantially parallel with one another. The plurality of micro-channels are communicated with the recycling chamber.

In some embodiments, the divergent channel has a front section, a middle section and a rear section. The front section is closest to the entrance terminal, the rear section is closest to the exit terminal, and the middle section is located between the front section and the rear section. The outlet chamber is communicated with the front section.

In some embodiments, depths of the front section, the middle section and the rear section of the divergent channel gradually increase along a direction from the entrance terminal to the exit terminal. A depth increase of the middle section along the direction is greater than depth increases of the front section and the rear section along the direction.

In some embodiments, widths of the middle section and the rear section of the divergent channel gradually increase along the direction from the entrance terminal to the exit terminal.

In some embodiments, the sperm sorter further includes a leading channel. The leading channel is communicated between the entrance terminal of the divergent channel and the swim up sorting chamber.

According to some embodiments, a sperm sorting method includes: providing a sperm sorter, wherein the sperm sorter comprises an inlet chamber, a swim up sorting chamber, a divergent channel, a recycling chamber and an outlet chamber, the swim up sorting chamber is communicated between the inlet chamber and the divergent channel, an entrance terminal and an exit terminal of the divergent channel are respectively communicated with the swim up sorting chamber and the recycling chamber, a depth and a width of the entrance terminal of the divergent channel are respectively less than a depth and a width of the exit terminal of the divergent channel, the outlet chamber is communicated with a portion of the divergent channel between the entrance terminal and the exit terminal; adding a culture medium into the sperm sorter via the swim up sorting chamber, and sealing the outlet chamber; adding a semen sample into the sperm sorter via the inlet chamber; opening the outlet chamber when a fluid height of the swim up sorting chamber and the recycling chamber is higher than a fluid height of the outlet chamber, such that sperms with high motility flow into the outlet chamber; and collecting the sperms with high motility from the outlet chamber.

In some embodiments, the sperm sorting method further includes: collecting living sperms from a top portion of the swim up sorting chamber.

As above, the sperm sorter according to some embodiments of the present disclosure is a passive sorting device, and performs sperm sorting by utilizing the characteristic behavior of sperms. Specifically, the sperm sorter integrates the swim up sorting chamber and the divergent channel. The swim up chamber performs sperm sorting by utilizing the swim-up behavior of sperms, whereas the divergent channel performs sperm sorting by utilizing the swim-against-flow behavior of sperms. In this way, the sperm sorter is capable of collecting sperm groups that have different amount and different motility ranges, for different artificial fertilization applications. In some embodiments, the divergent channel is a three-dimensional divergent channel. In other words, the divergent channel expanses both horizontally and vertically toward its exit terminal. As compared to a two-dimensional divergent channel, the three-dimensional divergent channel according to some embodiments of the present disclosure may have a greater volume. Therefore, more sperms can be sorted each time in the sperm sorter. Furthermore, in some embodiments, by disposing the filtering structure at the inlet terminal of the sperm sorter, a problem that the sperm sorter being jammed by the impurities of the semen samples can be avoided. Furthermore, in the sperm sorting method according to some embodiments of the present disclosure, a difference in fluid heights of the outlet chambers with respect to other portions of the sperm sorter can be formed by simply controlling whether the outlet chambers are sealed by the tape. As a result of this fluid height difference, the sperms with high motility in the divergent channel can be driven to move into the outlet chambers.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is an explosive diagram illustrating a sperm sorter according to some embodiments of the present disclosure.

FIG. 2 is a schematic cross-sectional diagram along line A-A′ in FIG. 1.

FIG. 3 is an enlarged schematic diagram illustrating the leading channel, the divergent channel and the outlet channel shown in FIG. 1.

FIG. 4 is an enlarged schematic diagram further illustrating the leading channel, the divergent channel, the outlet channel and the block according to some embodiments of the present disclosure.

### DESCRIPTION OF THE EMBODIMENTS

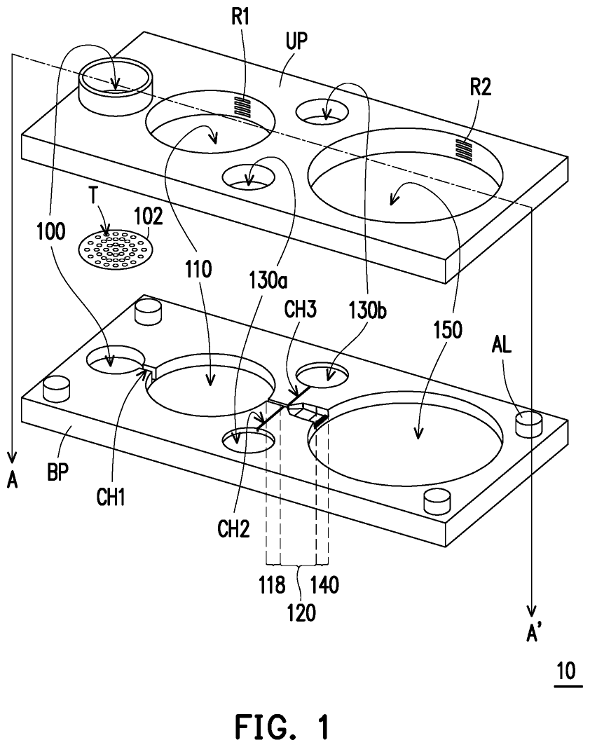

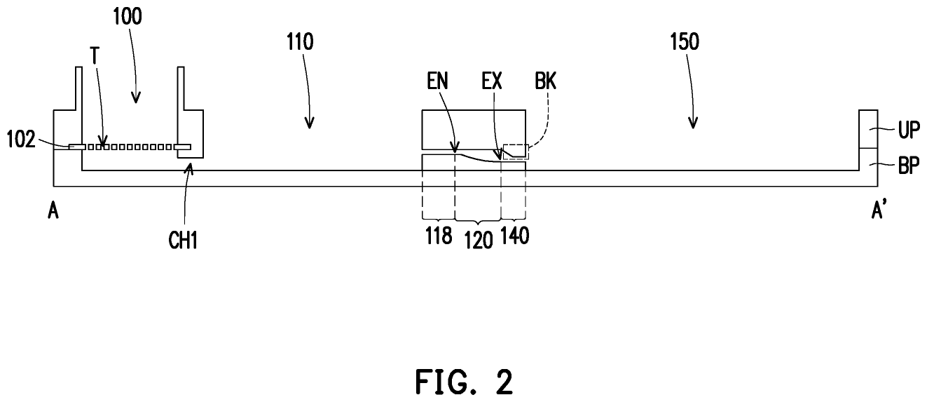

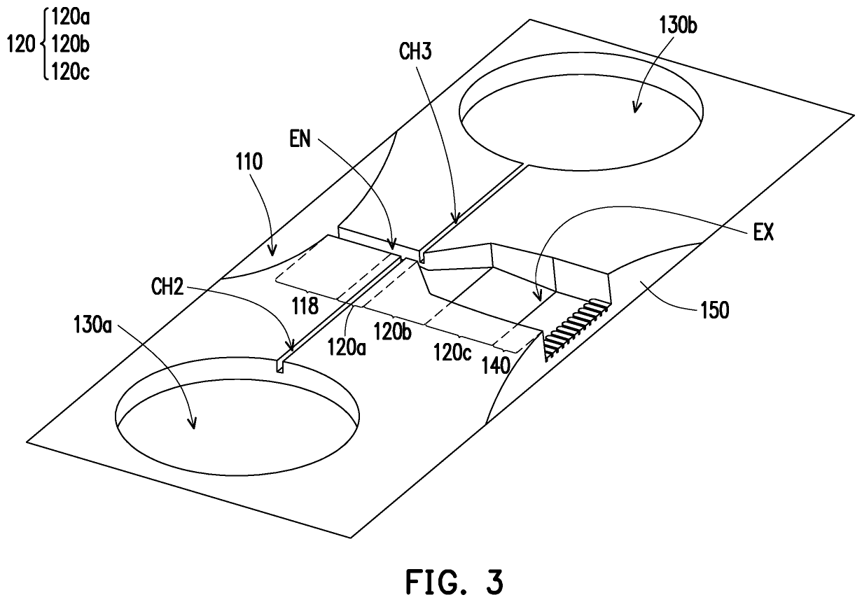

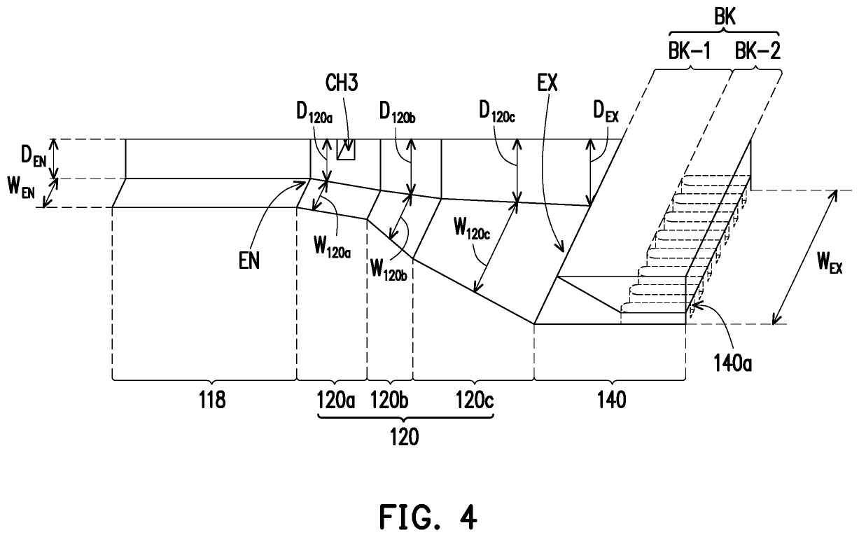

FIG. 1 is an explosive diagram illustrating a sperm sorter 10 according to some embodiments of the present disclosure. FIG. 2 is a schematic cross-sectional diagram along line A-A′ in FIG. 1. FIG. 3 is an enlarged schematic diagram illustrating the leading channel 118, the divergent channel 120 and the outlet channel 140 shown in FIG. 1. FIG. 4 is an enlarged schematic diagram further illustrating the leading channel 118, the divergent channel 120, the outlet channel 140 and the block BK according to some embodiments of the present disclosure.

Referring to FIG. 1, in some embodiments, the sperm sorter 10 may be an assembly of a top plate UP and a bottom plate BP. Materials of the top plate UP and the bottom plate BP may respectively include polymer material, glass, metal, semiconductor material or so forth, and may be identical or different from each other. In some embodiments, one or more sets of alignment structures AL may be formed at the surfaces of the top plate UP and the bottom plate BP that are facing each other. Each set of alignment structures AL may be a pair of male and female parts (e.g., one with a protrusion portions and another one with a recess portion). For instance, four sets of alignment structures AL at four corners of the sperm sorter 10 are depicted in FIG. 1. However, those skilled in the art may modify the locations and amount of the alignment structures AL according to design requirements, the present disclosure is not limited thereto.

Referring to FIG. 1 and FIG. 2, the sperm sorter 10 includes an inlet chamber 100. A semen sample may be temporarily stored in the inlet chamber 100, and may be fed into other portions of the sperm sorter 10 from the inlet chamber 100. In some embodiments, the inlet chamber 100 may include an upper part and a lower part. These upper part and lower part are respectively formed in the top plate UP and the bottom plate BP, and may be assembled together to form the inlet chamber 100. In some embodiments, the inlet chamber 100 is in a cylinder-like shape. In these embodiments, a diameter of the inlet chamber 100 ranges from 5 mm to 15 mm. In addition, the upper part of the inlet chamber 100 may protrude from a top surface of the top plate UP (i.e., a surface of the top plate UP that is opposite to the bottom plate BP). However, those skilled in the art may modify the shape and dimension of the inlet chamber 100 according to design requirements, the present disclosure is not limited thereto.

In some embodiments, the sperm sorter 10 further includes a filtering structure 102. The filtering structure 102 is disposed in the inlet chamber 100, and is functioned to filter the semen sample entering the inlet chamber 100. The filtering structure 102 may be a thin film with multiple through holes T. For instance, this thin film may include a SU8 dry film. In addition, the through holes T may be arranged in an array, or may be randomly distributed in this thin film. For instance, an aperture of the through hole T may range from 15 μm to 40 μm, or from 15 to 100 μm. In some embodiments, the filtering structure 102 may be disposed between the top plate UP and the bottom plate BP. After combining the top plate UP and the bottom plate BP, the filtering structure 102 could be located between the upper and lower parts of the inlet chamber 100. Impurities in the semen sample may be blocked from flowing through the filtering structure 102, and kept above the filtering structure 102. On the other hand, portions of the semen sample without impurities could enter the lower part of the inlet chamber 100 through the filtering structure 102.

The sperm sorter 10 further includes a swim up sorting chamber 110 communicated with the inlet chamber 100. In some embodiments, the semen sample may enter a swim up sorting chamber 110 after passing through the filtering structure 102. A swim up characteristic can be observed on sperms having motility greater than a certain level (or referred as living sperms). Therefore, the semen sample in the swim up sorting chamber 110 may actively form layers. The living sperms would float at an upper portion of the fluid in the swim up sorting chamber 110, whereas died sperms would form sediments in a lower portion of the fluid in the swim up sorting chamber 110. In some embodiments, motility and amount of the living sperms floating in the upper portion of the fluid in the swim up sorting chamber 110 would meet the requirement of the intrauterine insemination (IUI) method of artificial fertilization. For instance, the IUI method requires living sperms of more than 20000, or more than 5 million. In this way, the upper portion of the liquid in the swim up sorting chamber 110 could be collected for the IUI application when the recycling chamber 150 and outlet chamber 103a and 103b, which will be described in the following paragraphs, are sealed by tape. It should be noted that, in the present disclosure, motility of sperms is referred as moving velocity of sperms.

In some embodiments, the swim up sorting chamber 110 may include an upper part and a lower part. These upper part and lower part are respectively formed in the top plate UP and the bottom plate BP, and may be assembled together to form the swim up sorting chamber 110. In addition, a channel CH1 may be formed in the bottom plate BP, and is communicated between the lower part of the inlet chamber 100 and the lower part of the swim up sorting chamber 110. In some embodiments, the swim up sorting chamber 110 is in a cylinder-like shape. In these embodiments, a diameter of the swim up sorting chamber 110 ranges from 10 mm to 50 mm. However, those skilled in the art may modify the shape and dimension of the swim up sorting chamber 110 according to design requirements, the present disclosure is not limited thereto. Moreover, in some embodiments, a plurality of scotches R1 may be formed at a sidewall of the upper part of the swim up sorting chamber 110. The scotches R1 may extend along a horizontal direction, and arranged along a vertical direction. It could be easier for an operator to observe the fluid height in the swim up sorting chamber 110 with the help of the scotches R1.

The sperm sorter 10 further includes a divergent channel 120. The divergent channel 120 is communicated with the swim up sorting chamber 110, and the swim up sorting chamber 110 is communicated between the inlet chamber 100 and the divergent channel 120. In some embodiments, the divergent channel 120 is a trench disposed at a top surface of the bottom plate BP. After the top plate UP and the bottom plate BP are assembled together, a bottom surface of the top plate UP could define a top surface of this trench (i.e., the divergent channel 120). The divergent channel 120 has an entrance terminal EN close to the swim up sorting chamber 110 and an exit terminal EX away from the swim up sorting chamber 110. In some embodiments, the entrance terminal EN of the divergent channel 120 may be directly communicated with the swim up sorting chamber 110. In addition, as shown in FIG. 2, a bottom end of the entrance terminal EN of the divergent channel 120 may be higher than a bottom surface of the swim up sorting chamber 110 by about 0.5 mm to 5 mm. In this way, the upper portion of the fluid in the swim up sorting chamber 110 may be allowed to flow into the divergent channel 120. In other words, the living sperms in the swim up sorting chamber 110 may enter the divergent channel 120. Furthermore, referring to FIG. 3 and FIG. 4, a width W_{EN} and a depth D_{EN} of the entrance terminal EN of the divergent channel 120 may be respectively less than a width W_{EX} and a depth D_{EX} of the exit terminal EX of the divergent channel 120. For instance, the width W_{EN} may range from 0.1 mm to 2 mm, and the depth D_{EN} may range from 0.1 mm to 2 mm. On the other hand, the width W_{EX} may range from 1.5 mm to 10 mm, and the depth D_{EX} may range from 1 mm to 3 mm. In other words, the divergent channel 120 laterally and vertically fans out from the entrance terminal EN to the exit terminal EX. In this way, a flow rate in the divergent channel 120 may decrease toward the exit terminal EX. Sperms with high motility tend to move upstream, against the flow. In other words, the sperms with high motility would return toward the entrance terminal EN of the divergent channel 120. Since the flow rate in the divergent channel 120 may decrease toward the exit terminal EX, the sperms with high motility may be avoided from being washed to the exit terminal EX by the flow in the divergent channel 120. Therefore, the sperms with high motility may gather in the front half of the divergent channel 120 that is close to the entrance terminal EN, whereas sperms with low motility may flow to the exit terminal EX of the divergent channel 120.

Referring to FIG. 3 and FIG. 4, in some embodiments, the divergent channel 120 has a front section 120a, a middle section 120b and a rear section 120c. The front section 120a is closest to the entrance terminal EN, the rear section 120c is closest to the exit terminal EX, and the middle section 120b extends between the front section 120a and the rear section 120c. In some embodiments, the front section 120a extends from the entrance terminal EN to a side of the middle section 120b, whereas the rear section 120c extends from another side of the middle section 120b to the exit terminal EX. In some embodiments, a length of the front section 120a ranges from 1 mm to 10 mm, a length of the middle section 120b ranges from 1 mm to 10 mm, and a length of the rear section 120c ranges from 1 mm to 15 mm. In addition, a width W₁₂₀ₐ and a depth D₁₂₀ₐ of the front section 120a slightly increase toward the middle section 120b, and minimum values of the width W₁₂₀ₐ and the depth D₁₂₀ₐ are respectively equal to the width W_{EN} and the depth D_{EN} of the entrance terminal EN. A width W_{120b} and a depth D_{120b} of the middle section 120b greatly increase toward the rear section 120c. On the other hand, a width W_{120c} and a depth D_{120c} of the rear section 120c continuously increase toward the exit terminal EX, to be eventually equal to the width W_{EX} and the depth D_{EX} of the exit terminal EX, respectively. Therefore, the front section 120a of the divergent channel 120 has a relatively high flow rate, whereas the middle section 120b and the rear section 120c have relatively low flow rates. Accordingly, the fluid came from the swim up sorting chamber 110 may fluently enter the divergent channel 120, and may gradually slow down in the middle section 120b and the rear section 120c. The sperms with high motility may return to the front section 120a, and may respectively flow into outlet chambers 130a and 130b (as shown in FIG. 3) via a channel CH2 and a channel CH3 communicated with the front section 120a.

In some embodiments, a depth increase in the middle section 120b is greater than a depth increase of the front section 120a and a depth increase of the rear section 120c. For instance, the depth increase of the front section 120a is greater than 0.1 mm, and less than or equal to 1 mm. The depth increase of the middle section 120b may range from 0.1 mm to 2 mm. The depth increase of the rear section 120c may range from 0.1 mm to 1.5 mm. Moreover, a width increase of the middle section 120b may be greater than a width increase of the front section 120a and a width increase of the rear section 120c. For instance, the width increase of the middle section 120b may range from 0.1 mm to 10 mm, whereas the width increase of the rear section 120c may range from 0.1 mm to 5 mm. In these embodiments, the flow rate of the divergent channel 120 significantly decreases at the middle section 120b.

Referring to FIG. 3 and FIG. 4, in some embodiments, the sperm sorter 10 further includes a leading channel 118. The leading channel 118 is communicated between the entrance terminal EN of the divergent channel 120 and the swim up sorting chamber 110. In some embodiments, a width and a depth of the leading channel 118 are substantially constant along the extending direction of the leading channel 118, and are respectively equal to the width W_{EN} and the depth D_{EN} of the entrance terminal EN of the divergent channel 120.

Referring to FIG. 1 and FIG. 3, the sperm sorter 10 further includes the outlet chambers 130a and 130b communicated with the divergent channel 120. In some embodiments, the outlet chambers 130a and 130b are communicated with the front section 120a of the divergent channel 120, and are configured to collect the high motility sperms that have returned to the front section 120a of the divergent channel 120. In these embodiments, the outlet chambers 130a and 130b are respectively communicated with the front section 120a via the channels CH2 and CH3. In some embodiments, the outlet chamber 130a is closer to the entrance terminal EN of the divergent channel 120 than the outlet chamber 130b. In general, among the sperms that have returned toward the entrance terminal EN, the sperms with higher motility may end up being closer to the entrance terminal EN. Thereby, in these embodiments, the motility of the sperms collected at the outlet chamber 130a may be slightly higher than the motility of the sperms collected at the outlet chamber 130b. For instance, the sperms collected at the outlet chamber 130a are capable of moving against the flow with a flow rate (or referred as curvilinear velocity (VCL)) not less than 100 μm/s, or with a flow rate not less than 180 μm/s. Therefore, the sperms collected from the outlet chamber 130a may be available for the artificial fertilization method of intracytoplasmic sperm injection (ICSI) or in vitro fertilization (IVF). On the other hand, the sperms collected from the outlet chamber 130b are capable of moving against the flow with a flow rate (or referred as VCL) greater than 70 μm/s, or with a flow rate (VCL) ranging from 120 μm/s to 180 μm/s. In addition, an amount of these sperms is greater than or equal to 2000, or ranging from 50000 to 100000. Thus, the sperms collected from the outlet chamber 130b are available for the artificial fertilization method of in vitro fertilization (IVF). In addition, in some embodiments, the outlet chambers 130a and 130b may be disposed at opposite sides of the divergent channel 120. It should be noted that, two outlet chamber are illustrated, but those skilled in the art may adjust the amount of the outlet chamber(s) according to design requirements, the present disclosure is not limited thereto.

Referring to FIG. 3 and FIG. 4, in some embodiments, the sperm sorter 10 further includes an outlet channel 140 and a block BK. The outlet channel 140 is communicated with the exit terminal EX of the divergent channel 120. In some embodiments, the outlet channel 140 is a trench disposed at a top surface of the bottom plate BP. A width and a depth of this trench are substantially equal to the width W_{EX} and the depth D_{EX} of the exit terminal EX of the divergent channel 120, respectively. On the other hand, the block BK may be a protrusion portion protruded from a bottom surface of the top plate UP. After the top plate UP and the bottom plate BP are assembled together, a bottom surface of the block BK may define a top surface of the outlet channel 140. Viewing from another angle, the block BK may be regarded as a structure extending from a top surface of the outlet channel 140 into the outlet channel 140. In some embodiments, the block BK may include a first portion BK-1 that is relatively close to the divergent channel 120 and a second portion BK-2 that is relatively away from the divergent channel 120. A thickness of the first portion BK-1 gradually increases along a direction away from the divergent channel 120. On the other hand, a thickness of the second portion BK-2 is substantially constant, and equal to the maximum thickness of the first portion BK-1. In these embodiments, an end of the block BK close to the divergent channel 120 has a relatively small thickness, whereas another end of the block BK away from the divergent channel 120 has a relatively large thickness. Died sperms or the sperms with low motility may flow below the block BK, and exit the outlet channel 140.

In some embodiments, the outlet channel 140 includes a plurality of flow chocking micro-channels 140a, which are substantially parallel with one another. Died sperms or the sperms with low motility may pass through the flow chocking micro-channels 140a. In some embodiments, the flow chocking micro-channels 140a may be located below the second portion BK-2 of the block BK. The flow chocking micro-channels 140a may be functioned to further reduce the flow rate in the divergent channel 120, thus the sperms with sufficient motility are more likely to return to the front section 120a of the divergent channel 120. In some embodiments, a spacing between adjacent flow chocking micro-channels 140a may range from 0.05 mm to 1 mm, or from 0.1 mm to 1 mm.

Referring to FIG. 1 and FIG. 2, the sperm sorter 10 further includes a recycling chamber 150. The recycling chamber 150 is communicated with the exit terminal EX of the divergent channel 120, and died sperms or the sperms with low motility may flow into the recycling chamber 150. In some embodiments, the recycling chamber 150 may be communicated with the exit terminal EX of the divergent channel 120 through the outlet channel 140. In these embodiments, as shown in FIG. 3, the recycling chamber 150 may be communicated with the flow chocking micro-channels 140a. Regarding configuration of the sperm sorter 10, the outlet chambers 130a and 130b may be located between the swim up sorting chamber 110 and the recycling chamber 150. In addition, in some embodiments, the recycling chamber 150 may include an upper part and a lower part. The upper part and lower part are respectively formed in the top plate UP and the bottom plate BP, and may be assembled together to form the recycling chamber 150. In some embodiments, the recycling chamber 150 is in a cylinder-like shape. In these embodiments, a diameter of the recycling chamber 150 ranges from 10 mm to 80 mm, and the diameter of the recycling chamber 150 may be greater than the diameter of the swim up sorting chamber 110. However, those skilled in the art may modify the shape and dimension of the recycling chamber 150 according to design requirements, the present disclosure is not limited thereto. Moreover, in some embodiments, a plurality of scotches R2 may be formed at a sidewall of the upper part of the recycling chamber 150. The scotches R2 may extend along a horizontal direction, and arranged along a vertical direction. It could be easier for an operator to observe the fluid height in the recycling chamber 150 with the help of the scotches R2.

As above, the sperm sorter 10 according to some embodiments of the present disclosure is a passive sorting device, and performs sperm sorting by utilizing the characteristic behavior of sperms. Specifically, the sperm sorter 10 integrates the swim up sorting chamber 110 and the divergent channel 120. The swim up chamber 110 performs sperm sorting by utilizing the swim-up behavior of sperms, whereas the divergent channel 120 performs sperm sorting by utilizing the swim-against-flow behavior of sperms. In this way, the sperm sorter 10 is capable of collecting sperm groups that have different amount and different motility ranges, for different artificial fertilization applications. In some embodiments, the divergent channel 120 is a three-dimensional divergent channel. In other words, the divergent channel 120 expanses both horizontally and vertically toward the exit terminal EX. As compared to a two-dimensional divergent channel, the three-dimensional divergent channel 120 according to some embodiments of the present disclosure may have a greater volume. Therefore, more sperms can be sorted each time in the sperm sorter 10. Furthermore, in some embodiments, by disposing the filtering structure 102 at the inlet terminal of the sperm sorter 10, a problem that the sperm sorter 10 being jammed by the impurities of the semen sample can be avoided.

A sperm sorting method according to some embodiments of the present disclosure will be described with reference to FIG. 1 through FIG. 4.

Firstly, the sperm sorter 10 as described with FIG. 1 through FIG. 4 is provided. Thereafter, culture medium is added to the swim up sorting chamber 110. In some embodiments, the culture medium may include solutions of phosphate buffered saline (PBS), F10 (Ham's F-10) or the like. The culture medium may flow to every part of the sperm sorter 10, and rinse the sperm sorter 10. In some embodiments, sufficient volume of the culture medium is added into the sperm sorter 10, such that a fluid height of the inlet chamber 100 substantially reaches the filtering structure 102. Afterwards, the outlet chambers 130a and 130b may be sealed by a tape.

A semen sample is then fed to the sperm sorter 10 via the inlet chamber 100. After being filtered by the filtering structure 102, the semen sample may sequentially enter the swim up sorting chamber 110, the divergent channel 120, the outlet channel 140 and the recycling chamber 150 of the sperm sorter 10. Since the outlet chambers 130a and 130b are currently sealed, thus sperms are blocked from entering the outlet chambers 130a and 130b. In this way, fluid heights of the outlet chambers 130a and 130b may be lower than a fluid height of other portions of the sperm sorter 10 (e.g., the swim up sorting chamber 110 and the recycling chamber 150).

The tape is removed to open the outlet chambers 130a and 130b when the fluid heights of the outlet chambers 130a and 130b are lower than the fluid height of other portions of the sperm sorter 10. For instance, the tape is removed when the fluid height of the swim up sorting chamber 110 is higher than the fluid heights of the outlet chambers 130a and 130b, for example, by 0.5 mm. As a result of the fluid height difference, the sperms with high motility in the divergent channel 120 (e.g., in the front section 120a of the divergent channel 120) may enter the outlet chamber 130a and the outlet chamber 130b. In some embodiments, an amount of the sperms to be collected from the outlet chamber 130a and the outlet chamber 130b may be adjusted by changing how much time the outlet chamber 130a and the outlet chamber 130b are opened. Thereafter, the fluid in the outlet chamber 130a and the outlet chamber 130b may be collected by, for example, a pipette. As described above, the fluid in the outlet chamber 130a and the outlet chamber 130b should include the sperms with high motility. In some embodiments, referring to FIG. 3, the outlet chamber 130a is closer to the entrance terminal EN of the divergent channel 120 than the outlet chamber 130b, and the motility of the sperms collected from the outlet chamber 130a may be slightly higher than the motility of the sperms collected from the outlet chamber 130b. In these embodiments, the amount and motility of the sperms collected from the outlet chamber 130a may meet requirements of the intracytoplasmic sperm injection (ICSI) method or the in vitro fertilization (IVF) method. On the other hand, the amount and motility of the sperms collected from the outlet chamber 130b may meet requirements of the in vitro fertilization (IVF) method.

In addition, living sperms in the upper portion of the fluid in the swim up sorting chamber 110 may be collected by, for example, a pipette. In some embodiments, the amount and motility of the sperms collected from the upper portion of the fluid in the swim up sorting chamber 110 may meet requirements of the intrauterine insemination (IUI) method.

In summary, the sperm sorter according to some embodiments of the present disclosure is a passive sorting device, and performs sperm sorting by utilizing the characteristic behavior of sperms. Specifically, the sperm sorter integrates the swim up sorting chamber and the divergent channel. The swim up chamber performs sperm sorting by utilizing the swim-up behavior of sperms, whereas the divergent channel performs sperm sorting by utilizing the swim-against-flow behavior of sperms. In this way, the sperm sorter is capable of collecting sperm groups that have different amount and different motility ranges, for different artificial fertilization applications. In some embodiments, the divergent channel is a three-dimensional divergent channel. In other words, the divergent channel expanses both horizontally and vertically toward its exit terminal. As compared to a two-dimensional divergent channel, the three-dimensional divergent channel according to some embodiments of the present disclosure may have a greater volume. Therefore, more sperms can be sorted each time in the sperm sorter. Furthermore, in some embodiments, by disposing the filtering structure at the inlet terminal of the sperm sorter, a problem that the sperm sorter being jammed by the impurities of the semen sample can be avoided. Furthermore, in the sperm sorting method according to some embodiments of the present disclosure, a difference in fluid heights of the outlet chambers with respect to other portions of the sperm sorter can be formed by simply controlling whether the outlet chambers are sealed by the tape. As a result of this fluid height difference, the sperms with high motility in the divergent channel can be driven to move into the outlet chambers.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A sperm sorter, comprising:
an inlet chamber;
a swim up sorting chamber, communicated with the inlet chamber;
a divergent channel, wherein the swim up sorting chamber is communicated between the inlet chamber and the divergent channel, wherein the divergent channel has an entrance terminal close to the swim up sorting chamber and an exit terminal away from the swim up sorting chamber, and a width and a depth of the entrance terminal are respectively less than a width and a depth of the exit terminal;
a recycling chamber, communicated with the exit terminal of the divergent channel; and
an outlet chamber, communicated with a portion of the divergent channel between the entrance terminal and the exit terminal.

2. The sperm sorter of claim 1, further comprising:
a filtering structure, disposed in the inlet chamber, wherein a semen sample entering the inlet chamber is filtered by the filtering structure before flowing into the swim up sorting chamber.

3. The sperm sorter of claim 1, further comprising:
an outlet channel, communicated between the exit terminal of the divergent channel and the recycling chamber; and
a block, disposed in the outlet channel.

4. The sperm sorter of claim 3, wherein the block protrudes from a top surface of the outlet channel into the outlet channel, and wherein a thickness of one end of the block close to the divergent channel is less than a thickness of another end of the block away from the divergent channel.

5. The sperm sorter of claim 3, wherein the divergent channel comprises a plurality of micro-channels, wherein the plurality of micro-channels are arranged to be substantially parallel with one another, and wherein the plurality of micro-channels are communicated with the recycling chamber.

6. The sperm sorter of claim 1, wherein the divergent channel has a front section, a middle section and a rear section, the front section is closest to the entrance terminal, the rear section is closest to the exit terminal, the middle section is located between the front section and the rear section, and the outlet chamber is communicated with the front section.

7. The sperm sorter of claim 6, wherein depths of the front section, the middle section and the rear section of the divergent channel gradually increase along a direction from the entrance terminal to the exit terminal, and wherein a depth increase of the middle section along the direction is greater than depth increases of the front section and the rear section along the direction.

8. The sperm sorter of claim 7, wherein widths of the middle section and the rear section of the divergent channel gradually increase along the direction from the entrance terminal to the exit terminal.

9. The sperm sorter of claim 1, further comprising a leading channel, communicated between the entrance terminal of the divergent channel and the swim up sorting chamber.

10. A sperm sorting method, comprising:
providing a sperm sorter, wherein the sperm sorter comprises an inlet chamber, a swim up sorting chamber, a divergent channel, a recycling chamber and an outlet chamber, the swim up sorting chamber is communicated between the inlet chamber and the divergent channel, an entrance terminal and an exit terminal of the divergent channel are respectively communicated with the swim up sorting chamber and the recycling chamber, a depth and a width of the entrance terminal of the divergent channel are respectively less than a depth and a width of the exit terminal of the divergent channel, the outlet chamber is communicated with a portion of the divergent channel between the entrance terminal and the exit terminal;
adding a culture medium into the sperm sorter via the swim up sorting chamber, and sealing the outlet chamber;
adding a semen sample into the sperm sorter via the inlet chamber;
opening the outlet chamber when a fluid height of the swim up sorting chamber and the recycling chamber is higher than a fluid height of the outlet chamber, such that sperms with high motility flow into the outlet chamber; and
collecting the sperms with high motility from the outlet chamber.

11. The sperm sorting method of claim 10, further comprising:
collecting living sperms from a top portion of the swim up sorting chamber.
